(19) United States
(12) Patent Application Publication
(10) Pub. No.: US 2024/0212864 A1
(43) Pub. Date: Jun. 27, 2024

(54) TEMPORAL MODELING OF NEURODEGENERATIVE DISEASES

(71) Applicant: PANACEL-MIL LTD., Ra'anana (IL)

(72) Inventors: Boaz LERNER, Mazkeret Batia (IL); Dan HALBERSBERG, Tel-Aviv (IL); Yaniv MALOWANY, Tel-Aviv (IL)

(21) Appl. No.: 17/767,901

(22) PCT Filed: Oct. 11, 2020
(86) PCT No.: PCT/IB2020/059543

§ 371 Date: Apr. 10, 2022

Related U.S. Application Data
(60) Provisional application No. 62/913,404, filed on Oct. 10, 2019.

(51) Int. Cl. G16H 50/70; G16H 50/20; G16H 50/30

(52) U.S. Cl. CPC G16H 50/70; G16H 50/20; G16H 50/30

(57) ABSTRACT

A computer implemented method and system operative to: 1) predict neurodegenerative disease (ND) progression through classification applied to patient-specific vectors formed from clustering information, mined disease deterioration patterns identified in patient disease deterioration sequences, and disease moments derived from patient temporal functionality measure data; and 2) stratify ND patient population in accordance with the patients' disease progression aligned by time duration from disease onset.

### BACKGROUND OF THE INVENTION

Many neurodegenerative diseases (NDs) are fatal and affect the human motor system with a highly uncertain pathogenesis. The inner workings and mechanisms of these diseases are mainly unknown, but recent progress aims at better understanding pathogenesis to enable extension of life expectancy and improvement in life quality of those afflicted. The medical condition of a patient is evaluated at clinic visits using functionality measure (FM) items describing physical functionalities such as speaking, writing, or walking. Measuring FM values at each clinic visit helps in understanding the patient's medical condition, tracks his disease deterioration, and improves assessment of the influence of treatment.

Typically, raw FM data is employed to cluster patients into functional groups as an attempt to generate a homogenous group of patients for identifying a predictable disease progression pattern and rate that can be used in drug development and facilitate patient acclimation in view of expected deterioration. The problem with such methods is that patient data commonly contains a lot of noise resulting from variations in FM scoring between doctors and irregular testing periods between patients. Such noise in the data obscures disease progression patterns that could be helpful in facilitating drug development and patient acclimation, as noted above.

Accordingly, there is a need to improve machine learning techniques to generate more accurate models enhance neurological disease prediction.

### BRIEF DESCRIPTION OF THE DRAWINGS

The subject matter regarded as the invention is particularly pointed out and distinctly claimed in the concluding portion of the specification. The invention is best understood in view of the accompanying drawings in which:

FIG. 1 is a block diagram of an integrated system for temporal modeling of ND patients, according to an embodiment;

FIG. 2 is a general flow chart depicting pattern mining, classification, and clustering schemes linked together for predicting disease progression and stratifying patients into groups, according to an embodiment;

FIG. 3 is a flow chart depicting processing steps employed in training a classifier in accordance with class-specific deterioration patterns mined from a training patient population, according to an embodiment;

FIG. 4 is a flow chart depicting processing steps employed in training a classifier in accordance with class-specific deterioration patterns mined from a training patient population with additional cluster-specific information, according to an embodiment;

FIG. 5 is a flow chart depicting processing steps employed in patient clustering through dynamic time warping (DTW), according to an embodiment;

FIG. 6 is a flow chart depicting processing steps employed in a clustering scheme employing aligned deterioration pattern (ADP), according to an embodiment; and

FIG. 7 is a depiction of two patient visit series highlighting identification of most similar disease durations employed by ADP clustering, according to an embodiment; and

FIG. 8 is a depiction of disease progression measured by FM values of three arbitrary patients with visits with different acquisition times from disease onset, number, and frequency;

FIG. 9 depicts deterioration of ALSFRS values for Speech versus acquisition time from disease onset averaged over patients grouped in seven clusters following the application of the ADP clustering.

FIG. 10 depicts how clustering can distinguish patient groups by their characteristics due to their speaking ability.

FIG. 11 depicts deterioration of ALSFRS values for Walking versus acquisition time from disease onset averaged over patients grouped in five clusters following the application of the ADP clustering;

FIG. 12 also depicts how clustering can distinguish patient groups by their characteristics due to their walking ability.

It will be appreciated that for the sake of clarity, elements shown in the figures may not be drawn to scale and reference numerals

### DETAILED DESCRIPTION

In the following description, specific details are set forth in order to facilitate understanding of the invention; however, it should be understood by those skilled in the art that the present invention may be practiced without these specific details. Furthermore, well-known methods, procedures, and components have not been omitted to highlight the invention.

Embodiments of the present invention are directed, inter alia, to a computer implemented modeling system for temporal prediction and stratification of a neurodegenerative disease (ND) progression. Without diminishing in scope, the discussion here within focuses on amyotrophic lateral sclerosis (ALS) and its associated functional rating scale (ALSFRS); however, it should be understood that the system and methods set forth herewith also apply to other NDs such as Parkinson's and Alzheimer's, for example.

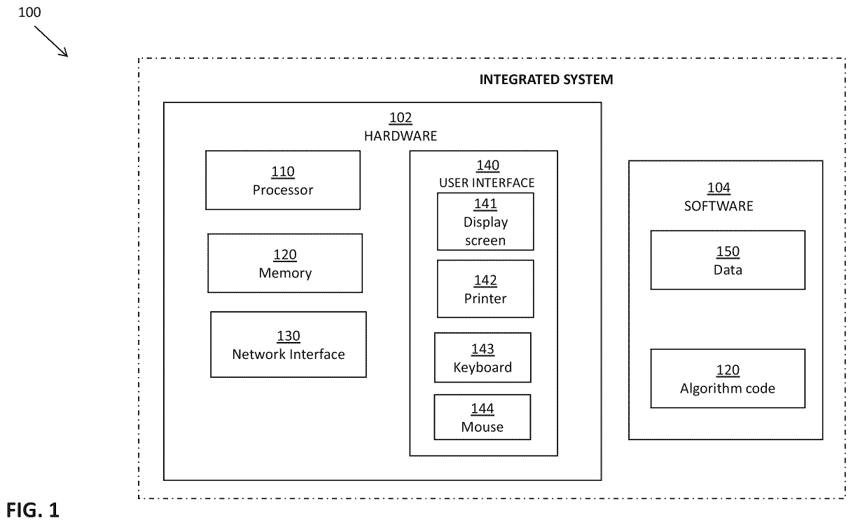

Turning now to the figures, FIG. 1 is a schematic block diagram of an embodiment of an integrated system 100 for personalized stratification and prediction of ND progression.

Integrated system 100 includes at least one processor 110 operative to execute one or more code sets, memory 120 operative to store the code sets and various data types, a network interface 130 enabling network functionality, user interface devices 140 like display screen 141, printers 142, keyboard 143, mouse 144, plus other user interface accessories.

As shown, system 100 includes a software module 104 including a database 150 of various types of patient data and a module of algorithm code 120 operative to process patient data. Code 120 must be executed by processor 110.

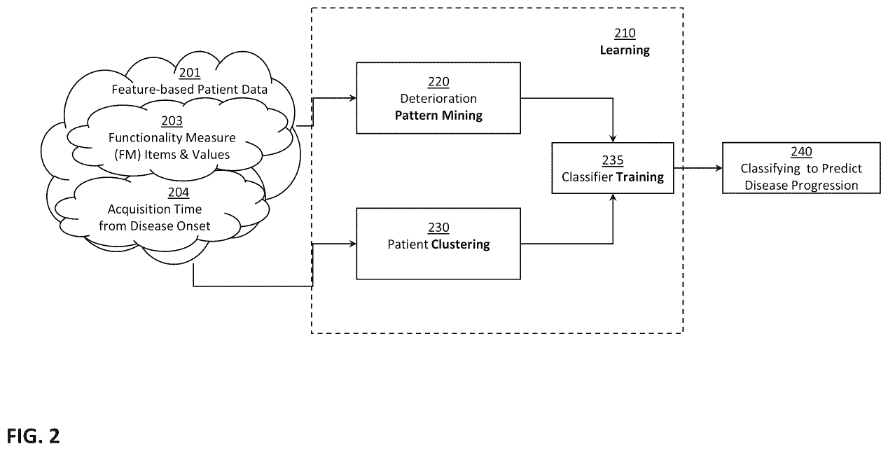

FIG. 2 is a general flow chart depicting temporal modeling schemes for predicting disease progression and stratifying a general patient population by employing three primary activities in learning stage 210: deterioration pattern mining 220, patient clustering 230, and classifier training 235, and then classifying 240, as will be further discussed. The modeling schemes are applied to feature-based patient data 201 by system 100 of FIG. 1. Feature-based patient data 201 including FM items and values 203, and their acquisition times measured from disease onset 204, and in some embodiments, other patient data like lab test results, demographics, and vital signs depending on the system embodiment. FM vary from one ND to another; ALS employs ALS ALSFRS (or ALSRFRS-R), Parkinson's disease employs unified Parkinson's disease rating scale (UPDRS) and Montreal-cognitive assessment (MoCA), and Alzheimer's disease employs Alzheimer's disease assessment scale (ADAS) (and ventricles volume), for example. Without diminishing in scope, this paper discusses ND modeling and prediction in the context of ALS, and thus FM will be represented by ALSFRS.

To this ends, feature-based patient data 201 is randomly split into training patient population (80%, for example) and testing (20%, for example).

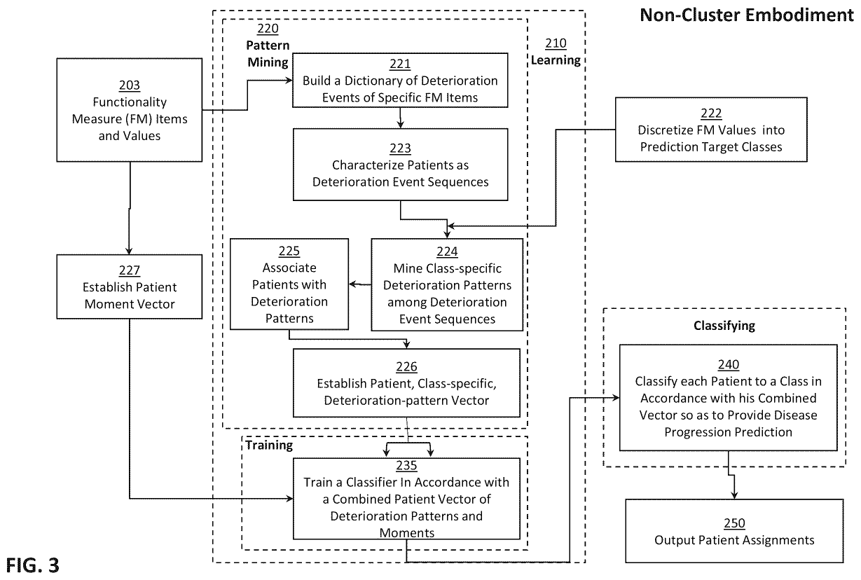

FIG. 3 is a flow chart depicting processing steps employed in training a classifier operative in accordance with mined deterioration patterns first identified among deterioration events of FM data of the above noted population data designated for training, according to an embodiment.

Specifically, a dictionary of specific deterioration events is built in step 221 from FM data 203 in which the specific deterioration events are each designated with an event label to be used during processing.

Table 1 below shows an example of multivariate patient data 203 and 204 with eleven visits (rows) of a single patient and values of ten ALSFRS functions (columns). The italic font represents a spurious deterioration (which should be ignored), and the bold font represents a real deterioration supported by the further visits.

TABLE 1
Sample FM Data of a Specified Patient using 11 Visits and 10 ALSFRS Functions

| Visit | | | | | Cutting | | Turning | | | | Time |
|---|---|---|---|---|---|---|---|---|---|---|---|
| number | Speech | Salivation | Swallowing | Writing | food | Dressing | in bed | Walking | Climbing | Respiratory | from onset |
| 1 | 4 | 4 | 4 | 4 | 4 | 3 | 3 | 2 | 1 | 4 | 0 |
| 2 | 4 | 4 | 4 | 4 | 4 | 2 | 3 | 2 | 1 | 4 | 90 |
| 3 | 4 | 3 | 4 | 4 | 4 | 4 | 3 | 2 | 1 | 4 | 180 |
| 4 | 4 | 4 | 4 | 4 | 4 | 3 | 3 | 2 | 1 | 4 | 240 |
| 5 | 4 | 4 | 4 | 4 | 3 | 3 | 3 | 2 | 1 | 4 | 360 |
| 6 | 4 | 4 | 4 | 4 | 4 | 3 | 2 | 2 | 0 | 4 | 450 |
| 7 | 4 | 3 | 3 | 3 | 1 | 1 | 2 | 1 | 0 | 4 | 630 |
| 8 | 4 | 3 | 3 | 3 | 1 | 1 | 2 | 1 | 0 | 4 | 780 |
| 9 | 3 | 3 | 3 | 2 | 1 | 0 | 1 | 1 | 0 | 4 | 900 |
| 10 | 3 | 3 | 3 | 2 | 1 | 0 | 1 | 1 | 0 | 4 | 990 |
| 11 | 3 | 2 | 2 | 2 | 1 | 0 | 1 | 1 | 0 | 4 | 1080 |

The dictionary of Table 2 below is used to transform each multi-dimensional sequence of Table 1 into a deterioration event sequence representation. Events in Table 2 represent deterioration of any of the ten ALSFRS items (events B-K) or none of them (A).

TABLE 2
Deterioration Event Dictionary

| Event ID | ALS functionality | direction |
|---|---|---|
| A | All | none |
| B | Speech | decrease |
| C | Salivation | decrease |
| D | Swallowing | decrease |
| E | Writing | decrease |
| F | Cutting food | decrease |
| G | Dressing | decrease |
| H | Turning in bed | decrease |
| I | Walking | decrease |
| J | Climbing stairs | decrease |
| K | Respiratory | decrease |

In step 223, system 100 (of FIG. 1) characterizes patients as deterioration events sequence by transforming the FM items of Table 1 into a sequence of events based on the event dictionary of Table 2. A sample deterioration event sequence representation is set forth below in Table 3.

TABLE 3
Sample Patient Deterioration Event Sequence Representation

| Visit Number | Items | Number of items |
|---|---|---|
| 2 | A | 1 |
| 3 | A | 1 |
| 4 | G | 1 |
| 5 | A | 1 |
| 6 | H, J | 2 |
| 7 | C, D, E, F, G, I | 6 |
| 8 | A | 1 |
| 9 | B, E, G, H | 4 |
| 10 | A | 1 |

It should be noted that visit 11 of Table 1 is not used for pattern mining (discussed in step 224) since it will be used later for the prediction of the next disease state, as will be further discussed.

Returning now to step 222, system 100 discretizes ALSFRS values in the range 0 to 40 into prediction target classes (e.g., low for 0-10, medium for 11-25, and high for 26-40) by applying piecewise aggregate approximation (PAA) and symbolic aggregate approximation (SAX) to reduce the target variable cardinality/dimension. Discretization facilitates detection of deterioration patterns unique to each discretized class, representing a distinct disease state to predict. It should be appreciated that additional classes can be set, generally in accordance with physician guidelines.

Other reasons to address each class separately are: 1) There is a higher chance that the class-specific patterns rather than patterns general to all ALSFRS values simultaneously will separate the classes, and 2) Unique deterioration sequences of classes of low populations will not achieve the occurrence threshold frequency needed to designate them as frequent deterioration patterns during mining compared with deterioration sequences of classes of high populations, thereby diminishing the effectiveness of future classification activities.

In step 224, system 100 mines for deterioration patterns among deterioration event sequences associated with patients of a population designated for classifier training by applying a sequential pattern mining algorithm (SPADE), according to an embodiment. In certain embodiments, PrefixSpan and other algorithms proving such functionality. Patterns are recognized and mined when achieving a threshold occurrence frequency. In a certain embodiment, the occurrence frequency is chosen as 0.3.

TABLE 4
Sample of Temporal Joining of SPADE

| SID | VID | ITEMS |
|---|---|---|
| 1 | 1 | B, C |
| 1 | 2 | C, D, E |
| 1 | 3 | F, H |
| 1 | 4 | D, H |
| 2 | 1 | B, D |
| 2 | 2 | E, H |
| 2 | 3 | C, F, H |
| 3 | 1 | B, D |
| 3 | 2 | F |
| 3 | 3 | F, H |
| 3 | 4 | C, D, E |
| 3 | 5 | B, D, E, F |

| | |
|---|---|
| B | C |

| SID | VID | SID | VID | . . . |
|---|---|---|---|---|
| 1 | 1 | 1 | 1 | . . . |
| 2 | 1 | 1 | 2 | |
| 3 | 1 | 2 | 3 | |
| 3 | 5 | 3 | 4 | |

B→

| SID | VID(B) | VID(C) | |
|---|---|---|---|
| 1 | 1 | 2 | . . . |
| 2 | 1 | 3 | |
| 3 | 1 | 4 | |

B→C→B

| SID | VID(B) | VID(C) | VID(B) |
|---|---|---|---|
| 3 | 1 | 4 | 5 |

Table 4 shows how SPADE mines patterns from sequential data. The left-hand side of Table 4 is the sequential database, where SID is the sequence ID (e.g., patient ID), VID is the visit ID, and ITEMS are the deterioration events associated with the visit (e.g., B and C are decreases in a patient's speech and salivation capabilities, respectively). The right-hand side of Table 4 demonstrates how SPADE grows sequence patterns by joining subsequences. For example, pattern B→C, i.e., a visit that includes event B followed by a visit that includes event C (right-top of Table 4), is a joining of Tables B and C (middle of Table 4) for the cases in which a patient has both events B and C, and event B occurred before event C. Similar, B→C→B (bottom of Table 4) is the joining of B to C→B. The support of the pattern is the number of rows (patients) that result from the joining, which is 3 in the case of B→C, for example. This method advantageously banishes the need to rescan the database multiple times because the sequence patterns are grown cumulatively.

In step 225, system 100 causes association between mined deterioration patterns and patients so that every patient of the population learned has at least one deterioration pattern.

In step 226, system 100 establishes patient vector embodying the mined class-specific, deterioration patterns, each represented as a binary variable indicating either its presence or nonpresence for this specific patient.

In step 227, system 100 establishes a second patient vector including moments of disease progression based on FM data 203 and acquisition time data 204. Moments of disease progression include disease progression rate, characterized in terms of FM deterioration per time unit, median, average, and minimum or maximum, all in terms of general or specific FM values.

In step 235, system 100 trains a classifier in accordance with the patient vector including moment vector from step 227 and deterioration pattern emanating from step 226, together with true labels of target class derived from step 222, according to an embodiment. Classifier is configurable to automatically select the appropriate combination of moments and mined patterns of disease progression or to use one supplied by a user. In a certain embodiment, all vector information is combined prior to step 235.

Following, in Table 5, is a sample of a combined patient vector including the above noted types of information.

TABLE 5
Combined Patient Vector

| | | | | | | | | General Variables | | |
|---|---|---|---|---|---|---|---|---|---|---|
| Pattern | | Slope (Moment) | | | Previous visit | | | Last | Time since | Target Class |
| D->E | B->C | Speech | — | ALSFRS | Speech | — | ALSFRS | decreased | onset | ALSFRS |
| 0 | 1 | 1/990 | | 2/990 | 3 | | 29 | No | 90 | high |

In step 240, system 100 assigns one or more patients to the appropriate target class in accordance with the combined patient vector of each patient and the trained classifier of step 235, thereby enabling disease progression predication. After training, the classifier is tested using the feature-based patient testing data derived as discussed above to evaluate and further improve it. In a certain embodiment, a random forest (RF) classifier is employed, whereas in another embodiment, classifiers like XGBoost are employed. While step 240 is applied to patient training and testing data as above, it is mainly used for predicting disease progression of new patients.

In step 250, system 100 outputs the patient assignment through any of a variety of output devices. In certain embodiments, the outputs are 1) a patient expected disease progressions and 2) a patient deterioration patterns associated with the target class to which the patient was assigned.

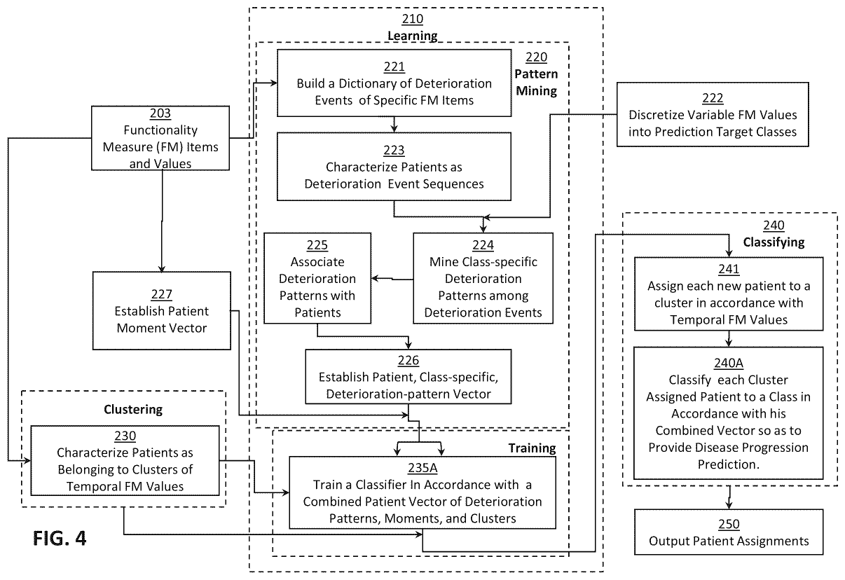

FIG. 4 is a flow chart depicting processing steps employed in training a classifier according to the processing steps set forth in FIG. 3 with additional clustering, according to an embodiment;

As shown, processing steps 203, 221-227 are the same as those set forth in FIG. 3.

In step 230, system 100 clusters patients in accordance with temporal FM values using various clustering schemes providing a patient a cluster identity to which he belongs as will be further discussed.

In step 235A, system 100 trains a classifier in accordance with a concatenated patient vector including moment vector from step 227, deterioration pattern emanating from step 226, and cluster identity from step 230 together with labels of true target class derived from step 222 across patient training data of a patient population. In a certain embodiment, an RF classifier is employed, whereas in another embodiment an XGBoost classifier is employed, whereas in yet in another embodiment other classifiers providing such functionality are employed.

In classifying stage 240, the clustering scheme derived in step 230 is used in step 241 to assign each new patient to a cluster in accordance with temporal FM values.

In step 240A, system 100 classifies each cluster assigned new patient to a class in accordance with his concatenated vector and the trained classifier of step 235A so as to provide disease progression prediction capacity.

In step 250, system 100 outputs the patient assignment through any of a variety of output devices. In certain embodiments, the outputs are 1) a patient expected disease progressions and/or 2) a patient deterioration patterns associated with the target class to which the patient was assigned.

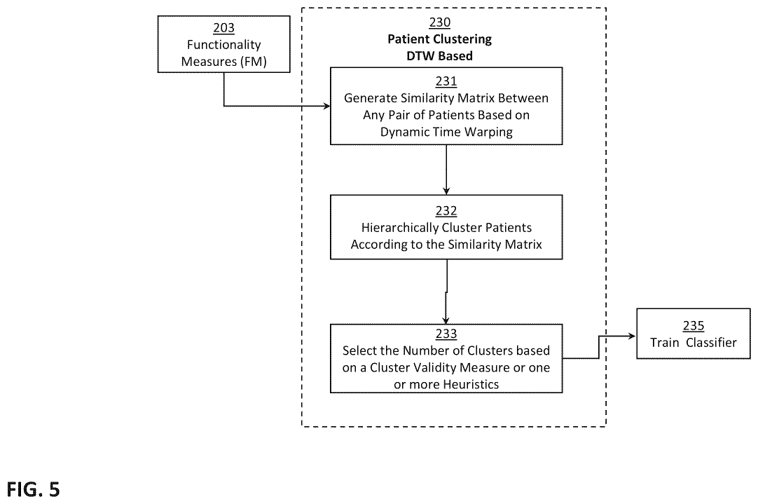

FIG. 5 is a flow chart depicting processing steps employed in patient clustering through dynamic time warping (DTW) and hierarchical clustering, according to an embodiment.

Dynamic time wrapping (DTW) measures the similarity (i.e. distance) between two temporal sequences such as those representing patient progressions. Table 6 shows patient five-class distribution stratified in four clusters. It can be seen that none of the clusters have a unique class, but each has one or two classes that are highly represented in it (e.g., Class 1 in Cluster 2). Therefore, the table demonstrates that the clustering did separate the patient population into more homogenous groups in term of target class distribution.

TABLE 6
The true (ALSFRS) class distribution per cluster

| | | | | | | Cluster |
|---|---|---|---|---|---|---|
| | Class 1 | Class 2 | Class 3 | Class 4 | Class 5 | size |
| Cluster 1 | 47% | 19% | 7% | 18% | 9% | 1,398 |
| Cluster 2 | 60% | 17% | 16% | 6% | 1% | 281 |
| Cluster 3 | 15% | 23% | 8% | 39% | 15% | 213 |
| Cluster 4 | 27% | 31% | 18% | 18% | 6% | 180 |

Table 7 depicts the average confusion matrix for the multiclass target variables (ALSFRS). A confusion matrix represents the classifier performance so that each entry in the matrix is the sum of samples that belong to class i and were classified as class j. For example, on average, 33.6 patients actually belong to class B, but were wrongly classified as class A. It can be seen in Table 7 that most errors are “mild” in terms of error severity (e.g., predicting A instead of B or vice versa), which is an advantage of our invention.

TABLE 7
RF average confusion matrix for the five-class classification

| | True Class (Y) | | | | | |
|---|---|---|---|---|---|---|
| | | A | B | C | D | E |
| Predicted | A | 210.4 | 33.6 | 3.2 | 0.7 | 0 |
| Class | B | 17.8 | 60.9 | 29.7 | 4.4 | 0 |
| (X) | C | 0.5 | 7.2 | 13.5 | 6.1 | 0 |
| | D | 1.2 | 6 | 13.6 | 64.4 | 8.1 |
| | E | 0 | 0.3 | 0 | 7.3 | 29.1 |

Table 8 summarizes the results in terms of accuracy, F1, and mean absolute error (MAE). The classifier accuracy is the percentage of sample that were correctly classified (the sum of the confusion matrix diagonal divided by the sum of the confusion matrix elements). F1 is a measure that considers both the precision and recall, where the precision is the number of correctly identified positive samples divided by the number of all positive samples, including those not identified correctly, and the recall is the number of correctly identified positive samples divided by the number of all samples that should have been identified as positive. Finally, the MAE measures the error severity, i.e., the average sum of distances between the true values and the predicted ones.

Table 8 reveals that the results improve as we move from the first row (naïve classifier) to the third row— clustering (recall that clustering include the pattern mining component). Although the differences with respect to accuracy, F1, and MAE are not statistically significant, a nonparametric Wilcoxson signed rank test (with a 0.05 confidence level) shows that our proposed invention (including pattern mining and clustering) is superior to the naïve and LSTM classifiers with respect to minor-class accuracy. The average accuracy improvement of our proposed invention over the baseline naïve classifier (with respect to the minor-class accuracy) was 80% (10.11% vs. 17.57%). Moreover, albeit the LSTM is usually ranked above the naïve classifier, it is always inferior to our enhanced invention (pattern mining and clustering) with respect to all measures.

TABLE 8
Accuracy, F1, and MAE for four experiments

| | Experiment | Accuracy | F1 | MAE |
|---|---|---|---|---|
| ALS | 1 - naïve classifier | 71.98 | 0.66 | 0.31 |
| | 2 - pattern mining | 72.87 | 0.67 | 0.30 |
| | 3 - clustering | 73.03 | 0.68 | 0.30 |
| | 4 - LSTM | 72.97 | 0.65 | 0.32 |

In step 231, system 100 generates a similarity matrix for any pair of patients of the training population based on DTW between FM data 203 of these patients.

In step 232, system 100 hierarchically clusters paired patients according to the similarity matrix. This clustering is implemented through complete-linkage hierarchical clustering or other algorithms providing such clustering functionality.

In step 233, system 100 selects the number of clusters maximizing a cluster validity

measure or one or more heuristics. Examples of suitable heuristics include Davies-Bouldin index, silhouette, or classification accuracy. As shown, the cluster output is used in classifier training in step 235A.

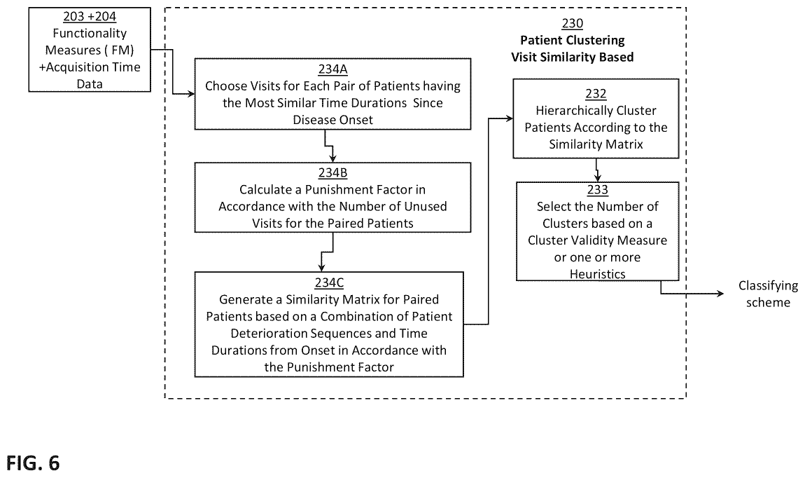

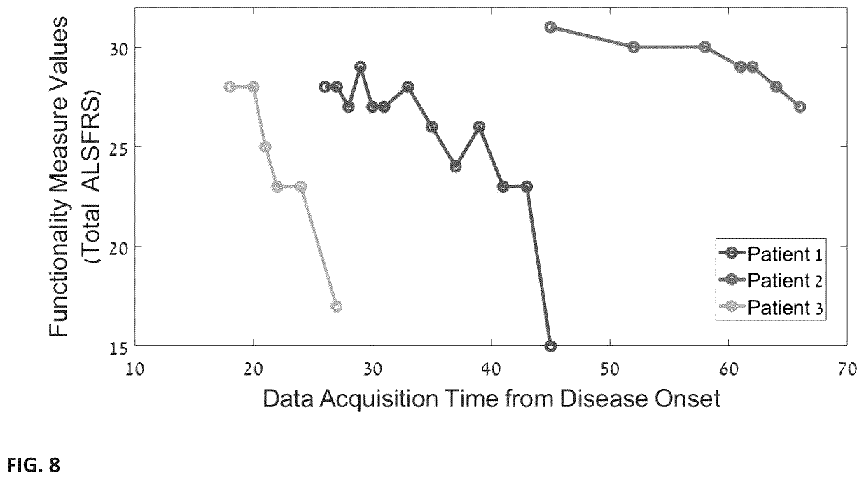

FIG. 6 is a flow chart depicting processing steps employed in patient clustering through aligned deterioration pattern (ADP) clustering that employs an additional provision to improve clustering accuracy, as clustering is an unsupervised procedure. Typically, temporal clustering is implemented on the basis of utilizing the complete patient disease progression sequences available. These sequences are usually started in different acquisition times from patient onset because each patient may be diagnosed in a different stage of his disease and/or change a doctor at some point, thus data of his previous visits are missing in the records and patient data analysis commences from the current doctor. In addition, the number of visits and their frequency are usually different among patients, which undermine correct comparison of disease progression as depicted in FIG. 8 demonstrates.

To address this problem, ADP advantageously identifies patient visit times of any two patients from a patient population to determine which visits are the closest in time in terms of disease duration from its onset. These patient visits embody FM data acquisition dates. Disease duration time from onset is determined for patient visits when FM data is acquired. Accordingly, patient similarity is first determined based on disease duration time and then measured in terms of their duration times and disease progressions. Identifying a common disease duration constitutes an advance in patient stratification in neurodegenerative patients and generally in other applications because it focuses on a patient representation based only on visit sequences aligned with respect to onset time when comparing a pair of patients instead of representations that are based on sequences of visits having different acquisition times, number, and frequency for the paired patients.

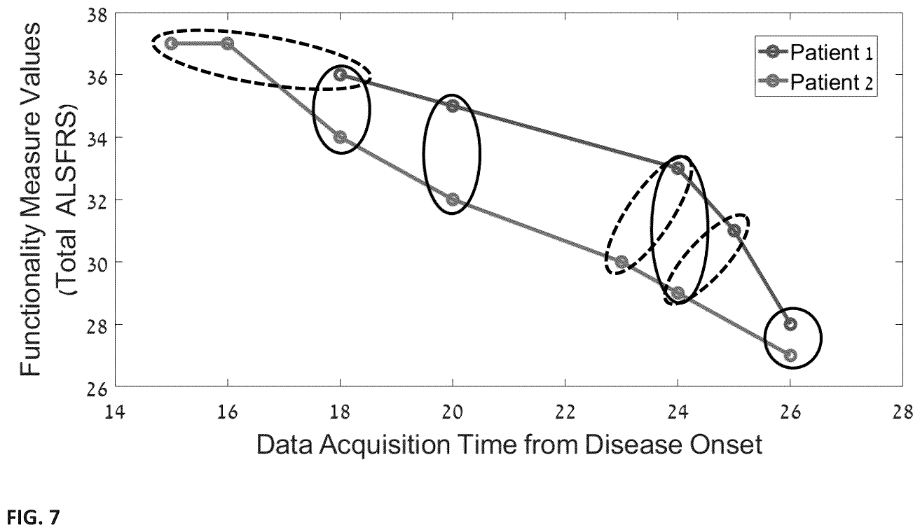

In step 234A, system 100 chooses visits between each pairs of patients having closest disease durations from onset; i.e., the most similar visits in terms of durations from the beginning of the disease for these patients in order to further compare the patients using visits that correspond to similar disease onsets, as depicted in FIG. 7.

This selection of visits is determined by the following seven stages of Pseudocode 1 according to an embodiment and also depicted in FIG. 7.
1) Select a pair of patients.
2) Initialize a two-dimensional list of visits to compare for this pair of patients
3) Calculate for the first visit of a patient in this pair the time interval to all visits of the other patient in the pair, repeat this procedure also to the other patient, considering all calculated time intervals of both patients, identify the shortest one, identify the patient of the pairs that his first visit yielded the shortest time interval and the corresponding visit of the other patient in order to compare between the patients using the first visit of the patient and the corresponding visit of the other patient.
4) Update the list of visits of this pair of patients with the two selected visits.
5) Temporarily remove for the sake of comparison of this pair all visits (if any) of the other patient that precede his corresponding visit as well as those visits that were selected for the comparison and updated the list of visits for this pair of patients.
6) Repeat the process in Stages 3-5 until no visits of either of the patients exist.
7) Repeat for all pairs of patients.

Following is sample of code implementing step 234A of finding the nearest patient visits between patients i and j in a certain embodiment.
TSOᵢ is {TSO_{i,1}, . . . . , TSO_{i,m}} times since onset of m visits of patient i.
Xᵢ is {X_{i,1}, . . . . , X_{i,m}} FM values in the m visits of patient i.

Algorithm 1: Find nearest visits between pstients i and j

```
Input: TSOᵢ, TSOⱼ
Output: C ∈ [symbol]  , D ∈ [symbol]
Initialization: X = TSOᵢ, Y = TSOⱼ, C = Ø, D = Ø
        While X and Y not empty do
            x = TSO_{i,1}, y = TSO_{j,1}
            [y_{candidate}y_{location}] = argmin _{y′∈Y} {d(x,y′)} % looking for the similar visit in object j
                                                        vector to the first visit of
                                                                  object i
            [x_{candidate},x_{location}] = argmin _{y′∈X} {d(x′,y)}
          If (d(x,y_{candidate}) < d(y,x_{candidate}))
            C ← x, D ← TSO_{j,y}_{_{location}}
            X = X\x
            Y = Y\{y,....,y_{candidate}}
          Else
            C ← TSO_{i,x}_{_{location}} , D ← y
            X = X\{x,....,x_{candidate}}
            Y = Y\y
          End
        End
```

In step 234B, system 100 calculates a punishment factor in accordance with the number of unpaired visits for the paired patients. In this manner, the punishment factor effectively reduces the reliability of similarity measured for two patients having paired visits in accordance with the number of unpaired visits.

In step 234C, system 100 calculates a similarity matrix for paired patients based on a combination of patient deterioration sequences and durations from onset in accordance with the punishment factor. The punishment factor is implemented by increasing a distance between the patients having paired visits in accordance with the number of their unpaired visits. The increased distance will effectively reduce the similarity of the two patients during generation of a similarity matrix.

In step 232, system 100 hierarchically clusters all patients according to the similarity matrix as set forth above in the context of FIG. 5.

In step 233, system 100 selects the number of clusters maximizing/minimizing a cluster validity measure or one or more heuristics as set forth above in the context of FIG. 5.

FIG. 7 depicts implementation of step 234A, according to an embodiment. Patient 1 time since onset visits (in months) are: {18,20,24,25,26}, and Patient 2 time since onset visits are (in months): {15,16,18,20,23,24,26}. These visits will characterize the following example. Distance between visits is measured according to the Euclidean distance.

Following Pseudocode 1, in Stage 1, Patients 1 and 2 are selected. In Stage 2, the list of visits is initialized to compare Patients 1 and 2. In Stage 3, 15 is the first visit of Patient 2, and 18 is the first visit of Patient 1, and these visits “compete” to be the first to initialize the comparison between the two patients. Following the calculation from 15 to all Patient 1's visits and from 18 to all Patient 2's visits, we show below the minimal distances for the two patients. Solid and dashed ellipses in FIG. 7 represent some of the closest visits to Patient 1's first visit and Patient 2's first visit, respectively:

$$1 ) abs ( 15 - 18 ) = 3$$

$$2 ) abs ( 18 - 18 ) = 0 ,$$

Because (<3, Patient 1 “wins” the competition, and in Stage 4, we update the list with (18,18), which are the first two visits to compare between the patients. In Stage 5, all the visits are removed until 18 for both patients. In Stage 6, this process is repeated for the remaining visits of the patients, and returns to Stage 1. The remaining visits for Patient 1 are: {20,24,25,26}, and the remaining visits for Patient 2 are: {20,23,24,26}. The minimal distances calculated for the new first visits are:

$$1 ) abs ( 20 - 20 ) = 0 ,$$

$$2 ) abs ( 20 - 20 ) = 0 ,$$

Because 0-0, these two visits are kept and update the list with (20,20) (Stage 4), before we temporarily remove these visits for this pair of patients (Stage 5). Stage 6 returns the algorithm to Stage 1 with visits {24,25,26} for Patient 1 and {23,24,26} for Patient 2. The minimal distances calculated for the new first visits are:

$$1 ) abs ( 24 - 24 ) = 0 ,$$

$$2 ) abs ( 23 - 24 ) = 1$$

Because 0<1, the list of visits is updated with (24,24) (Stage 4), previous visits are removed, and this procedure is continued until getting the final sequences ready for patient comparison which are {18,20,24,26} for Patient 1 and {18,20,24,26} for Patient 2.

After completing steps 234A and 234B, calculation of the distance between any pair of patients is implemented in accordance with the following equations of Algorithm 2:

$$1 ) FunctionDist ( i , j ) = \frac{1}{m} {\sum }_{k = 1}^{m} {( {X}_{i , k} - {X}_{j , k} )}^{2}$$

$$2 ) TimeDist ( i , j ) = {( \frac{1}{m} {\sum }_{k = 1}^{m} {TSO}_{i} - {TSO}_{j} )}^{2}$$

$$3 ) EuclideanDist = \frac{1}{V} {\sum }_{n = 1}^{V} \sqrt{FunctionDist + TimeDist}$$

$$4 ) W = \frac{V}{\frac{\vert {Visit}_{i} + {Visit}_{j} \vert}{2}}$$

$$5 ) Penalty = λ * ( 1 - W )$$

$$6 ) Dist = EuclideanDist + Penalty$$

$$V - Number of chosen visit$$

There is a need to consider both the functional difference in progressions of two patients and the differences in time of visits where progression is measured, meaning that it is insufficient that two patients will be similar according to their visits, but require that they be similar also at the times these visits span from disease onset. Because the visits are an unequal-length time-series, it is considered that, in comparison between patients, there can be large differences between the number of visits of Patients 1 and 2. Therefore, Algorithm 2 has two parts: The first part calculates the Euclidean distance (Equations 1-3) between patient combined representation by different functions (FM) and the times since onset when these functions were measured. Since there is a need to calculate the time since onset and the functional values with the same weights, z scaling is applied.

In the second part of Algorithm 2 (Equations 4-6), the distance measured between two patients is punished (increased) if it was achieved using only a small number of visits, demonstrating, in this case, an unreliable calculation. If, in contrast, all visits have been used in the calculation of the distance between two patients, the calculation is supposed to be reliable, and no punishment is exercised. Hyper-parameter lambda (2) in Equation 5 is set in advance to balance the contribution of the punishment factor to distance measured based, as above, on FM and times since onset of visit of the two compared patients compared with too many unpaired visits in the comparison.

It should be appreciated that the clustering scheme of FIG. 6 can also be applied in a wide variety of clustering applications in the absence of additional classifying operations according to the embodiment.

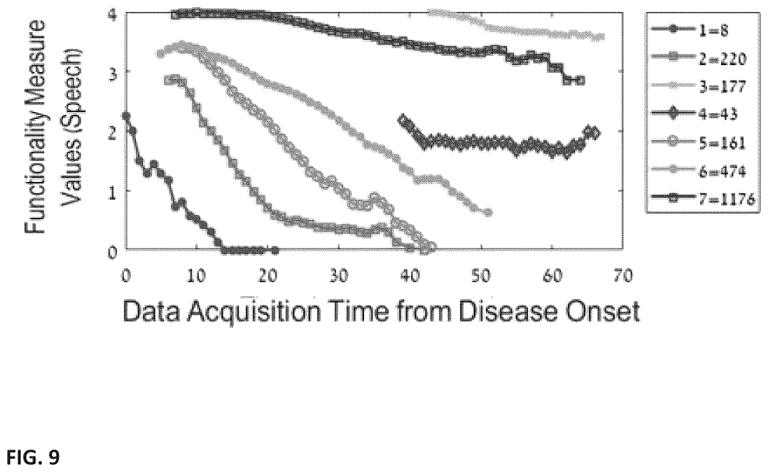

FIG. 9 depicts deterioration of ALSFRS values for Speech versus acquisition time from disease onset averaged over patients grouped in seven clusters following the application of the ADP clustering.

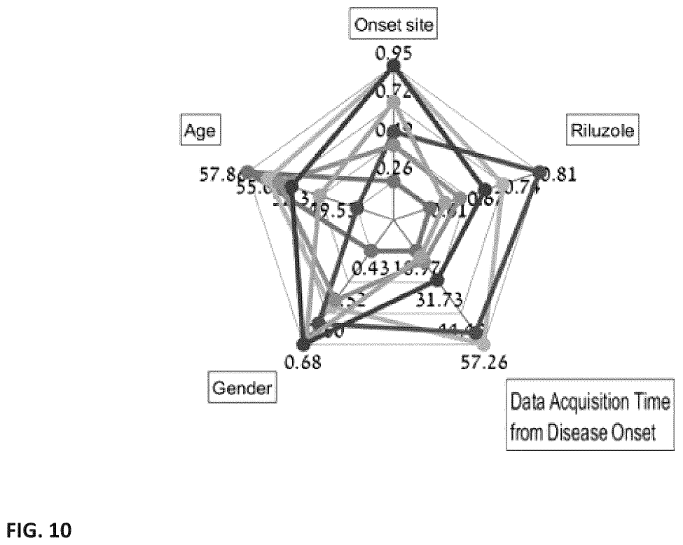

Based on the clusters identified, FIG. 10 depicts how clustering can distinguish patient groups by their characteristics due to their speaking ability. For example, and based on the same scheme of clusters (colors) as in FIG. 9, the yellow group is of relatively old patients for whom the disease started in the limbs and who deteriorate very slowly, whereas the orange group is of old patients, mostly women, who the disease started for them in the bulbar and their deterioration is very fast.

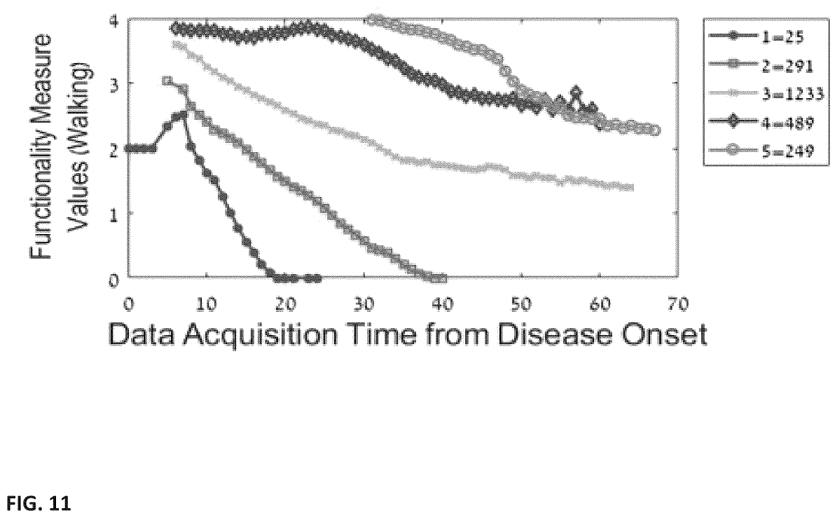

FIG. 11 depicts deterioration of ALSFRS values for walking versus acquisition time from disease onset averaged over patients grouped in five clusters following the application of the ADP clustering.

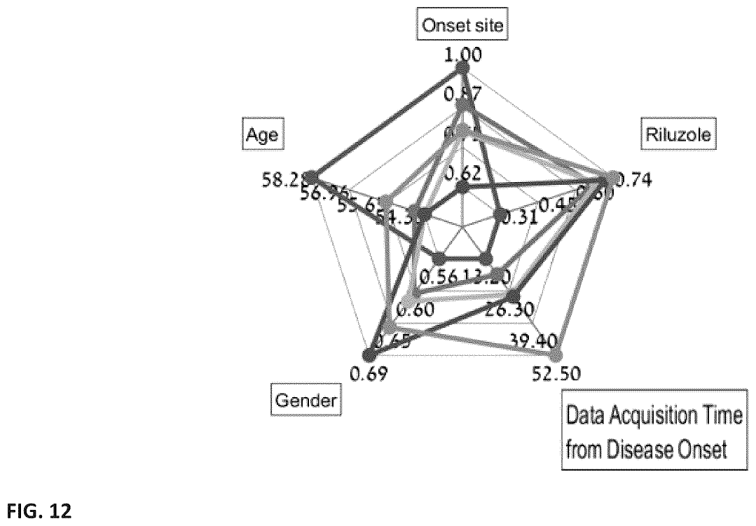

Based on the clusters identified, FIG. 12, similarly to FIG. 10, depicts how clustering can distinguish patient groups by their characteristics due to their walking ability. For example, and based on the same scheme of clusters (colors) as in FIG. 11, the purple group is of very young patients, almost all of them are women, for whom the disease mostly started in the bulbar and who deteriorate moderately in their walking ability, whereas the blue group is of old patients, mostly men, who the disease started for them in the limbs and their deterioration is very fast.

What is claimed is:

1. A method for predicting Neurodegenerative Disease (ND) progression performed on a computing device having a processor, memory, and one or more code sets stored in the memory and executed in the processor, the method comprising:
receiving feature-based patient data, functionality measure (FM) data for a plurality of ND patients of a patient population, the data including a series of FM values and corresponding acquisition dates for each of the ND patients, each of the FM acquisition dates characterizing a disease duration time from disease onset;
identifying patient deterioration events in the FM data;
characterizing patients as deterioration event sequences;
mining one or more class-specific, deterioration patterns from the patient deterioration event sequences, the patterns characterized by threshold occurrence frequency across patient deterioration event sequences of a training patient population;
causing association between each of a plurality of patients of the patient population and at least one of the deterioration patterns;
establishing a class-specific patient vector for each of the plurality of the patients of the training patient population, the patient vector including one or more deterioration patterns;
training a classifier to assign each of the plurality of the patients of the training patient population to a target class in accordance with each of the class-specific, patient vectors;
using the classifier to assign a new patient to one of the target classes; and
outputting disease prediction of the new patient in accordance with his assignment to one of the target classes.

2. The method of claim 1, wherein the patient vector includes one or more moments of disease progression.

3. The method of claim 1, wherein the classifier is further configured to assign a patient to a target class in accordance with patient clusters characterized by common temporal FM values.

4. The method of claim 3, wherein the patient clusters are established in accordance with Dynamic Time Warping (DTW).

5. The method of claim 2, wherein the patient clusters are established in accordance with aligned deterioration pattern (ADP) clustering; the method comprising:
matching FM acquisition dates of a first series and second series of FM acquisition dates from the FM data, the matching implemented in accordance with closest temporal proximity between FM acquisition dates of each of the first and the second series of FM acquisition dates;
generating a punishment factor in accordance with a number of unmatched FM acquisition dates in each of the first and the second series of FM acquisition dates; and
generating a similarity matrix for all patient pairs having the matching FM acquisition dates, in accordance with the FM values of each patient of the patient pairs and the punishment factor.

6. An aligned deterioration pattern (ADP) clustering method for stratifying Neurodegenerative Disease (ND) patient progression performed on a computing device having a processor, memory, and one or more code sets stored in the memory and executed in the processor, the method comprising:
receiving feature-based patient data, functionality measure (FM) data for a plurality of ND patients of a patient population, the data including a series of FM values and corresponding acquisition dates for each of the ND patients, each of the FM acquisition dates characterizing a disease duration time from disease onset;
matching FM acquisition dates of a first series and second series of FM acquisition dates from the FM data, the matching implemented in accordance with closest temporal proximity between FM acquisition dates of each of the first and the second series of FM acquisition dates;
generating a punishment factor in accordance with a number of unmatched FM acquisition dates in each of the first and the second series of FM acquisition dates;
generating a similarity matrix for all patient pairs having the matching FM acquisition dates, in accordance with the FM values of each patient of the patient pairs and the punishment factor;
hierarchically clustering the patient pairs having the matching FM values and acquisition dates into a dendrogram according to the similarity matrix;
deriving a clustering scheme based on the number of distinct clusters from the dendrogram;
assigning a new patient to one of the patient clusters most closely characterizing temporal FM values of the patient; and
outputting a resulting cluster assignment of the new patient.

7. The method of claim 6, wherein the deriving a clustering scheme is implemented by selecting a number of clusters derived from the dendrogram.

8. The method of claim 7, wherein the selecting a number of clusters derived from the dendrogram is implemented with cluster validity measure.

9. The method of claim 7, wherein the selecting a number of clusters derived from the dendrogram is implemented with one or more heuristics.
